(19) United States
(12) Patent Application Publication
(10) Pub. No.: US 2008/0148769 A1
(43) Pub. Date: Jun. 26, 2008

(54) Therapy pad used on a head

(76) Inventor: Muriel Higgins, Cape Coral, FL (US)

Correspondence Address: Muriel Higgins, 905 Sw Santa Barbara Place, Cape Coral, FL, 33991, US

(21) Appl. No.: 11/645,308

(22) Filed: Dec. 26, 2006

(51) Int. Cl. A61F 7/10

(52) U.S. Cl. 62/530

(57) ABSTRACT

A therapeutic device that applies cooling to areas of the forehead and the eyes of a patient in traumatized areas. The device consists of sleeve made of natural fabric that lies gently to the skin. The sleeve is compartmentalized to hold from one to four gel packs depending on a users need. The gel packs are composed of a non toxic substance that will remain unfrozen and malleable even when cooled to temperatures at which water filled packs would freeze solid. The individual packs readily conform to facial features due to the material used and thereby contributing to the improved comfort of the user. The gel packs can be heated for use by submerging them in hot water for use over sinus areas of the face. For facial use comfort can be had by allowing the user to be in a semi-inclined position rather than prone, without any restriction or pressure of a band going around the head of the user to cause undue compression to an already traumatized tissue.

### FIELD OF THE INVENTION

The invention pertains to the general field of medical devices and methods for cooling body parts to alleviate pain swelling and inflammation, in particular, a new method especially useful for cold applications post operatively as well as trauma to the upper face; one or both eyes.

### BACKGROUND OF THE INVENTION

U.S. Pat. No. 6.241,711 discloses a therapeutic face and eye mask which covers the whole face.

U.S. Pat. No. 5,628,772 is a therapeutic massage mask.

U.S. Pat. No. 5,984,951 illustrates an ice cooling medical device which can be applied to different locations on the body by way of straps.

Typically, a patient will hold an ice pack on a sore spot with one hand or will try to balance it in place while lying down, in either case, the person's ability to function or undertake various tasks is impaired and rest is difficult. Therefore, it would be very useful and desirable to have a cooling device that could be used in a semi-prone position rather than flat and not to have to hold an ice bag or similar device in place by hand, thus freeing the hand from holding something against his or her face.

It is a well known fact in the medical profession that cooling of body tissues is necessary immediately after physical trauma or injury to reduce swelling of such tissues. A variety of such ice packs and related devices are commercially to provide such therapy. Therapy of facial trauma, however, involves unique considerations. Human beings are much more sensitive in the areas of the face and the head than in other areas of the body. An application of direct pressure to the face, particularly after surgery, can cause the patient to suffer additional discomfort beyond that incurred by the surgery. Traditional ice packs posses characteristics that are disadvantageous when applied to a patient who has suffered facial trauma. The disadvantages in known in known ice packs are so great that medical practitioners have yearned for alternatives to traditional ice packs. Patients have been advised to place bags of frozen vegetables i.e. peas on their faces to achieve a cooling therapeutic effect.

### BRIEF DESCRIPTION OF THE INVENTION

An object of the invention is to provide a cooling therapy without the harsh effects of raw ice. This is accomplished by placing soft fabric next to the skin with a pliable cold gel pad located within a movable pocket that can be placed on top of various trauma affected areas without creating any significant or hardly any pressure at all.

### BRIEF DESCRIPTION OF THE DRAWINGS

FIG. 1 shows the therapeutic device as a laid out flat double sided sleeve;

FIG. 2 shows the therapeutic sleeve on the head of a patient in one use;

FIG. 3 shows the therapeutic sleeve on the head of a patient in a different use;

FIG. 4 shows the therapeutic device in a side view whereby both the forehead and the eyes are covered;

FIG. 5 is a frontal view of the therapeutic device covering both sides of the forehead and one eye.

### DETAILED DESCRIPTION OF THE INVENTION

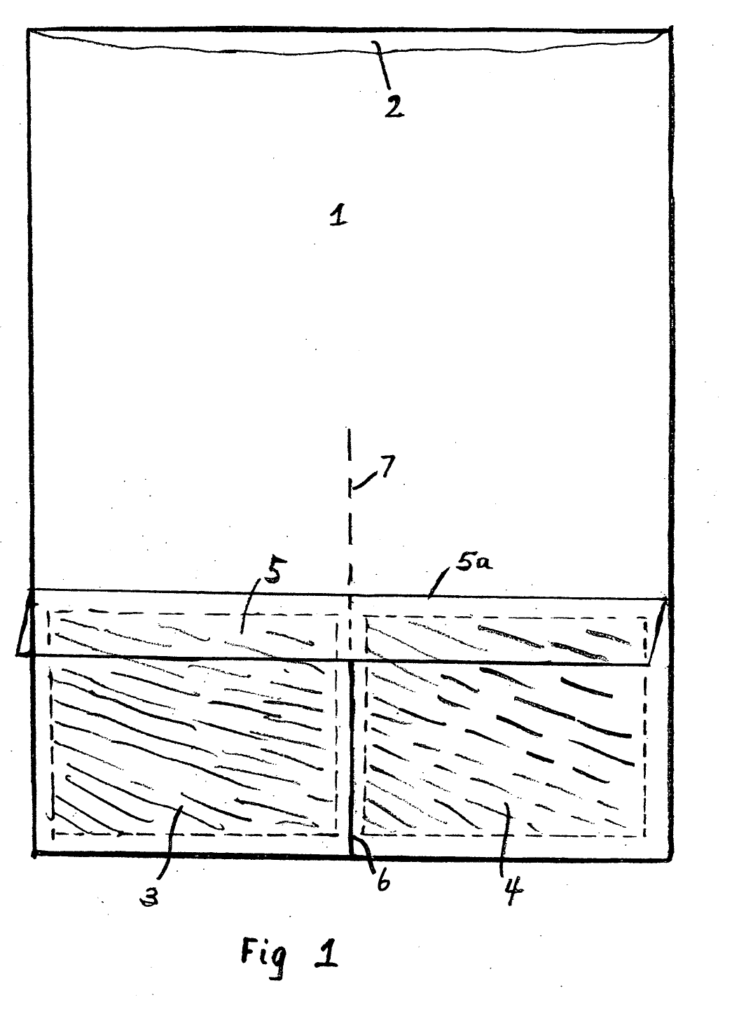

FIG. 1 shows the therapeutic device in the form of a double sided sleeve 1. The interior of the sleeve 1 is accessible through an opening in the top of the sleeve. The sleeve is subdivided into an upper and lower compartment by way of a seam 5a which anchors a flap 5 to the sleeve to cover the openings of two lower pockets 3 and 4. The pockets are separated from each other by a separation line 6 so that the pockets 3 and 4 can move independently from each other as will be explained below. The pockets are open at their tops but the opening is covered by way of the flap 5. In the center of the bottom of the upper compartment there is a sewn seam 7 which will thereby establish two separated compartment. In use, the sleeve 1 will be draped over the head of a patient. The shaded lines within the pockets are indicating the presence of at least two gel packs, which when frozen provide the necessary cold therapy to affected areas. The presence of the gel packs are outlined by dashed lines.

In this embodiment, when the sleeve is draped over the head of a patient and when correctly positioned the two pockets with each containing a gel pack therein would cover both eyes of the patient. Obviously, only one eye could be covered when so desired as will explained below.

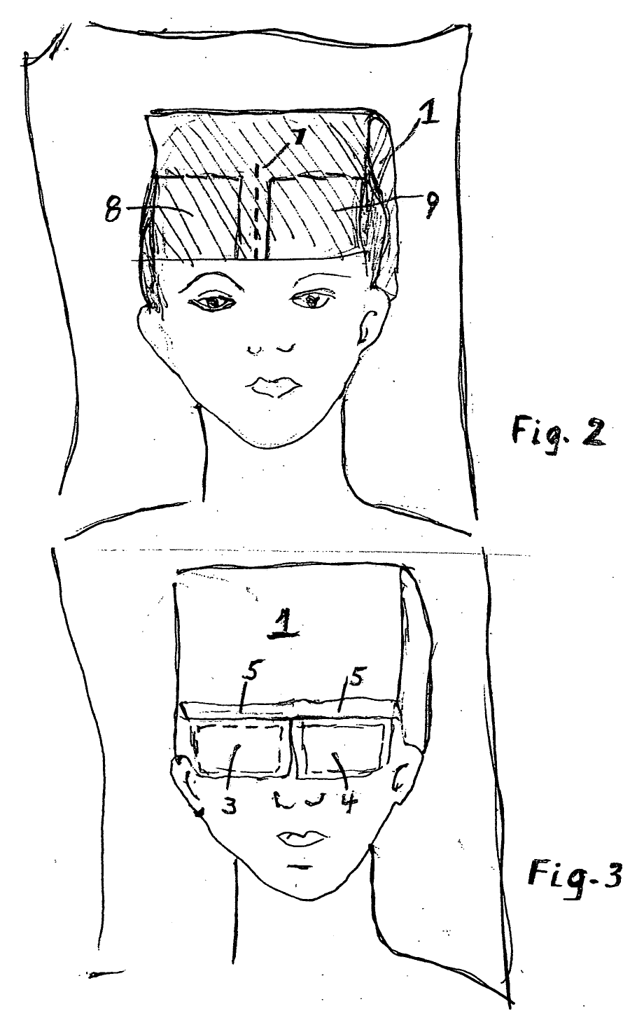

FIG. 2 shows a different embodiment of use. In this instance, two gel packs 8 and 9 are placed in the two compartment in the bottom of the upper compartment which are separated by the stitch line 7. The two pockets 3 and 4, designed to cover the eyes remain folded upwardly and not in use. The two gel packs 8 and 9 are now placed over the forehead to provide a therapeutic treatment in that area if a certain trauma is present there.

FIG. 3 shows still a different embodiment of use by way of a frontal view. In this embodiment the sleeve 1 again is draped over the head of the patient and both movable pockets 3 and 4 have a gel pack therein (outlined by dashed lines) and both movable pockets 3 and 4 are placed over the eyes of a patient. Also shown in this Fig. is the flap 5 which will cover the openings of the pockets.

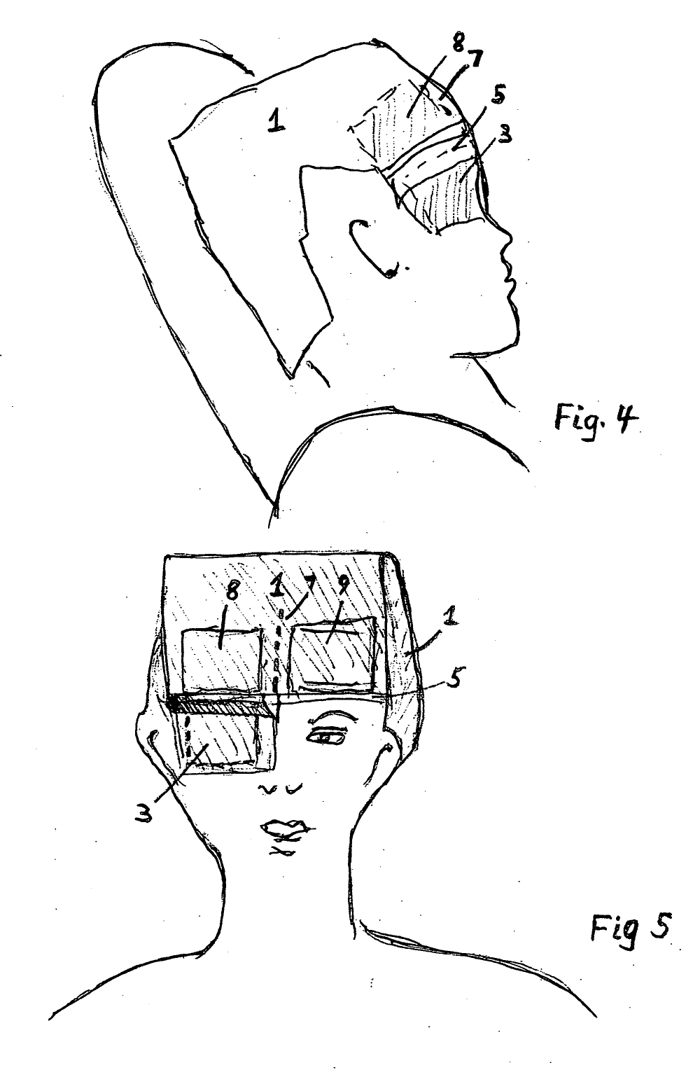

FIG. 4 Shows a side view of the sleeve 1 draped over the head of a patient In this embodiment of use there two upper gel packs each placed into the compartments 8 and 9 (only 8 is shown). In addition there are two gel packs placed each into the movable pockets 3 and 4 (only is shown). This arrangement would cover an instance where there is a full upper facial trauma.

FIG. 5 is still another use embodiment in a front view. In this embodiment there are two gel packs 8 and 9 covering the full frontal forehead while one gel pack is contained in the movable pocket 3 to only cover the left eye.

### Operation

From all of the above, it can now be seen that a vary versatile and useful therapeutic device has been disclosed. The versatility lies in the fact that any number of gel packs can be placed over areas that are affected by certain traumas.

The cotton sleeves need not be water proofed because the gel is contained in hermetically sealed packs The packs can be formed of two plastic layers that define a closed space and can be filled with any known cooling medium that include water, glycerin and hydroxethyl cellulose and others.

What I claim is:

1. A versatile therapeutic device to cover trauma on the forehead and the eyes of a patient, said device consisting of a fabric sleeve to be draped over the head and the front of a patient, said sleeve is made of two sheets of fabric sewn into said sleeve, on opening is placed at an upper end of said sleeve to permit access to the interior of said sleeve, a lower end of said sleeve is provided with two independent and movable pockets to receive cold gel packs therein, an intermediate seam divides said movable pockets from said sleeve leaving an upper compartment in said sleeve.

2. The therapeutic device of claim 1 including a flap covering an opening in each of the movable pockets.

3. The therapeutic device of claim 1 including a seam in said upper compartment half between its lateral edges to create at least two compartments to receive a cold gel pack in each of said compartments.
